(19) United States
(12) Patent Application Publication
(10) Pub. No.: US 2021/0030314 A1
(43) Pub. Date: Feb. 4, 2021

(54) A METHOD AND APPARATUS FOR QUANTIFYING VOLUME OF A DEPOSITED SKIN-PRINT

(71) Applicant: INTELLIGENT FINGERPRINTING LIMITED, Cambridge Cambridgeshire (GB)

(72) Inventors: Daniel O'Connor, Impington, Campbridge, Cambridgeshire (GB); Robert Homby, Impington, Cambridge, Cambridgeshire (GB)

(21) Appl. No.: 16/969,077

(22) PCT Filed: Feb. 13, 2019
(86) PCT No.: PCT/GB2019/050387

§ 371 Date: Aug. 21, 2020

(30) Foreign Application Priority Data
Feb. 13, 2018 (GB) 1802358.0
Feb. 14, 2018 (GB) 1802426.5

(51) Int. Cl. A61B 5/1172; A61B 5/00; G01B 11/06; G01B 9/02; G06K 9/00

(52) U.S. Cl. CPC A61B 5/1172; A61B 5/4833; G06K 9/00026; G01B 11/0675; G01B 9/0209; A61B 5/0077

(57) ABSTRACT

A method and apparatus for quantifying volume of a deposited skin-print A method of determining a volume of skin-print residue deposited on a substrate is disclosed. The method comprises: performing interferometry on the substrate with skin-print residue deposited thereon to determine raw topography data of the substrate including the skin-print residue. The method also comprises: processing the raw topography data including subtracting topography of the substrate without the skin-print residue from the raw topography data in order to determine skin-print residue topography data. The method further comprises: determining volume of skin-print residue deposited on the substrate from the skin-print residue topography data.

### BACKGROUND

An impression left by the friction ridges of human skin, such as the skin of a human finger, contains information regarding the identity of the human. It is widely known that the appearance of the impression of the human finger, known as a fingerprint, is unique to each human and may be used to confirm the identity of the human. The appearance of the impression of the skin of other human body parts may also be unique to each human and so may also be used to confirm the identity of the human. Impressions of human skin, including but not limited to the skin of the human finger, may be called skin-prints.

In addition to the appearance of the impression left by human skin, the impression may contain chemical species which themselves may be detected in order to obtain further information.

For example, when a human intakes a substance (e.g. by ingestion, inhalation or injection) the substance may be metabolised by the human body giving rise to secondary substances known as metabolites. The presence of a particular metabolite can be indicative of a specific intake substance. The intake substance and/or metabolites may be present in sweat and, as such, may be left behind in a skin-print, e.g. a fingerprint. Detection of such metabolites in a skin-print can be used as a non-invasive method of testing for recent lifestyle activity such as (but not limited to) drug use, or compliance with a pharmaceutical or therapeutic treatment regime.

Importantly, the taking of a skin-print is much simpler than obtaining other body fluids such as blood, saliva and urine, and is more feasible in a wider range of situations. Not only this but since the appearance of the skin-print itself provides confirmation of the identity of the person providing the skin-print, there can be greater certainty that the substance or substances in the skin-print are associated with the individual. This is because substitution of a skin-print, particularly a fingerprint, is immediately identifiable from appearance whereas substitution of, for example, urine, is not immediately identifiable from appearance. As such, testing for one or more substances in a skin-print provides a direct link between the one or more substances and the identity of the human providing the skin-print.

The applicant has demonstrated various techniques for chemical analysis of skin-prints, including the use of mass spectrometry, for example paper spray mass spectrometry. The applicant has also developed a lateral flow skin-print analysis technique as described in WO 2016/012812, published 28 Jan. 2016.

While in some circumstances it may be sufficient to provide a test which simply determines whether a quantity of an analyte of interest is above or below a specific threshold, in other circumstances it may be helpful to provide a quantitative result. This may be particularly applicable is situations where an acceptable quantitative threshold is defined by an independent standards agency. Such quantitative results and/or thresholds may, for example, be measured as mass of analyte per unit volume of skin-print.

Determining volume of skin-print deposited on a substrate may not be straightforward since the volume may be small to measure to a sufficient degree of precision.

Accordingly, a need exists for a technique for determining a volume of skin-print deposited on a substrate.

### STATEMENTS OF INVENTION

Against this background there is provided a method of determining a volume of skin-print residue deposited on a substrate, the method comprising:
performing interferometry on the substrate with skin-print residue deposited thereon to determine raw topography data of the substrate including the skin-print residue;
processing the raw topography data including subtracting topography of the substrate without the skin-print residue from the raw topography data in order to determine skin-print residue topography data;
determining volume of skin-print residue deposited on the substrate from the skin-print residue topography data.

This enables a reliable determination of a deposited skin-print having a volume of the order of nanolitres to tens of nanolitres. Furthermore, the determination is non-destructive and allows the skin-print to remain in situ for subsequent processing, meaning that the appearance of the skin-print (which provides a unique identifier of the skin-print provider) is not prejudiced.

### BRIEF DESCRIPTION OF THE DRAWINGS

FIG. 1 shows a typical grid pattern for segmenting a substrate on which a skin-print to be analysed may be deposited;

FIG. 2 shows threshold values for gradient of the substrate based on a segmentation approach like that shown in FIG. 1 for a clean substrate wherein threshold is selected such that 95% of surface topography is identified as substrate;

FIG. 3 shows, on the left, an image of the surface topography of a field of view containing skin-print, where colours represent different heights with non-measured points in white and shows, on the right, the same region having undergone the threshold analysis to distinguish substrate (white) from deposited skin-print residue (black);

FIG. 4 shows further processing of the data (right) by second order polynomial least squares form removal of points corresponding to the substrate;

FIG. 5 shows further processing of the data (right) when thresholded between 0.5% and 95.5%;

FIG. 6 shows further removal of background features achieved by subtracting a second order polynomial fit of the substrate from the entire surface;

FIG. 7 shows the production of a mask of salt and pepper noise from points that lie above or below the median filtered surface by more than 1.5 μm wherein the median filter uses a 3×3 pixel kernel;

FIG. 8 shows a surface immediately before (left) and after (right) non-measured point filling using smooth interpolation from the surrounding measured points; and

FIG. 9 shows areas are removed from the edges of this field of view which overlap with points in neighbouring fields of view based on the initial metadata processing.

### DETAILED DESCRIPTION

Specific embodiments of the disclosure will now be described by way of example only.

In the present disclosure, it should be noted that the term skin-print is used to refer to the residue of a deposited skin-print rather than to the constituents of the skin-print residue when present on the human skin. The skin-print contains sweat which may include both eccrine sweat and sebaceous sweat. It is envisaged that the technique of the present method is used to determine the volume of skin-print on a substrate other than the human body. In most cases, this is likely to be a substrate with a considerable degree of planarity. A glass slide or a plastic substrate may be appropriate.

Where the substrate has a degree of inherent flexibility such that planarity may be compromised, the substrate may be provided in a supporting structure to maintain its planarity. Such a supporting structure may be in the form of a cartridge.

Whether or not a cartridge provides a supporting structure to maintain planarity, a cartridge may protect the substrate from damage which might result in reduced planarity or surface imperfections that would make the subsequent analysis more complex.

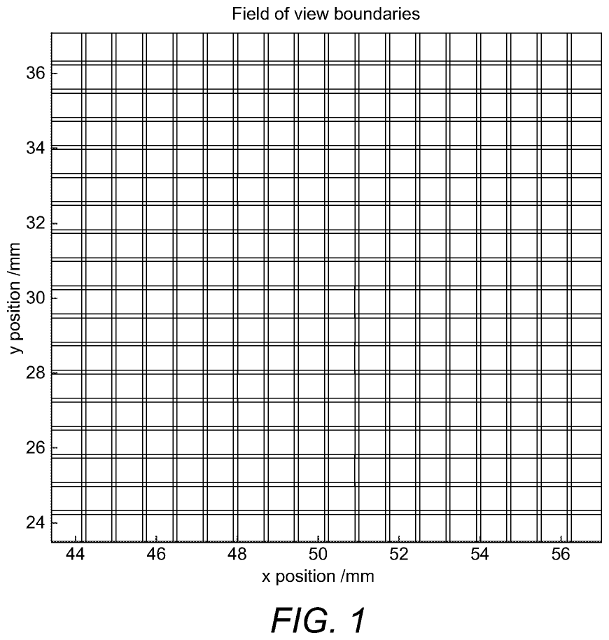

In one approach, a white light interferometer having a 0.835 mm by 0.835 mm field of view may be used. This is insufficient for determining the topography of a skin-print since a typical fingerprint, for example, may occupy an area in excess of 10 mm by 10 mm on a substrate. Accordingly, a grid approach may be adopted whereby a plurality of white light interferometry data may be obtained by performing an analysis of each grid area. In one approach, an 18×18 grid approach may be adopted, by which 324 scans may be performed. By employing a 5% overlap between adjacent scans to ensure unbroken coverage, a total scan area of 13.6 mm×13.6 mm is obtained. This may be sufficient to cover at least a meaningful proportion of the majority of fingerprints. FIG. 1 illustrates an implementation of an 18×18 grid arrangement with 5% overlap between grid areas.

The data obtained directly from a typical surface topography measuring instructed based on white light interferometry may be termed raw topographical data.

Data processing of the raw topographical data resulting from the white light interferometric scans may be necessary in order, among other things:
to distinguish between topography of the skin-print and topography of the underlying substrate;
to account for missing data points (for example, resulting from spikes in the surface whose gradient is such that the white light interferometry technique is unable to determine a data point);
to stitch the individual scan data for each field of view in order to determine the topography across the entire skin-print area; and
to integrate the volume between the upper surface of the underlying substrate and the top surface of the skin-print residue in order to arrive at a total result, measured in nano litres or tens of nano litres, or perhaps even hundreds of nano litres.

If each field of view gives rise to a ˜4 MB file, an 18×18 grid of views will give rise to a ˜1.3 GB file. In order to optimise processor and memory usage, it may be that each view is processed either in turn or in parallel, and only once the processing of each view is complete are the views stitched together.

As the skilled person would readily appreciate, the surface of the substrate on which the skin-print residue is deposited, will not be perfectly planar. Furthermore, it may be that a plane drawn through the average height of each surface point (that is, a perfect, virtual plane that best resembles a real but imperfect plane of the surface) has a non-zero gradient. In order to be able to determine the topography of the skin-print residue, it is necessary to determine the substrate topography so as to be able to ascertain the volume enclosed between the two, which equates to the volume occupied by the skin-print residue itself.

A number of techniques have been developed in order to achieve this objective. The aim of substrate background removal is to fit a suitable function to the form of the substrate and subtract it from the entire surface topography, ideally leaving the surface of the substrate as a horizontal plane through z=0 in the processed surface data.

In one arrangement, a threshold method is employed based on gradients of topography.

The first step in this process may be to segment the surface topography into two parts: the substrate and the skin-print features. To do this, a threshold method based on gradients of the topography may be used.

First, the magnitude of the gradient of the surface topography may be found, and then the magnitude of the gradient of those resulting points is thresholded to give the segmentation mask, effectively finding regions of the surface which are flat and smooth to within a chosen threshold.

In Matlab, this segmentation mask may be produced by

mask=imgradient(imgradient(points))<threshold;

where “points” is a matrix of uniformly spaced surface height data (with no lateral point spacing information), and “threshold” is the threshold which distinguishes fingerprint features from substrate.

Given a measurement of the substrate with no skin-print present, the threshold may be selected such that the substrate segmentation process identifies almost all of the substrate surface as part of the substrate, excluding severe imperfections.

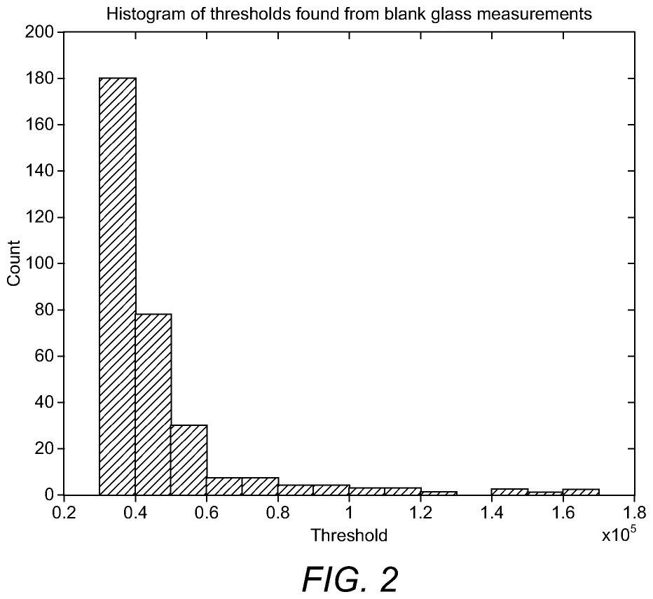

In one example approach, surface data from the 18×18 fields of view of a clean glass slide were processed and a segmentation threshold value was selected which identified 99.5% of the surface as being part of the substrate. The resulting 324 threshold values are shown in a histogram in FIG. 2. Fields of view of the glass which contained larger imperfections led to larger threshold values in order to reach the 99.5% inclusion level, leading to a long tail at high threshold values. The mean of all of these thresholds, 38393 points, was selected as the segmentation threshold.

This specific threshold value depends on the lateral point spacing and vertical resolution of the topography data. This needs to be accounted for when using a different WLI objective lens or different measurement settings.

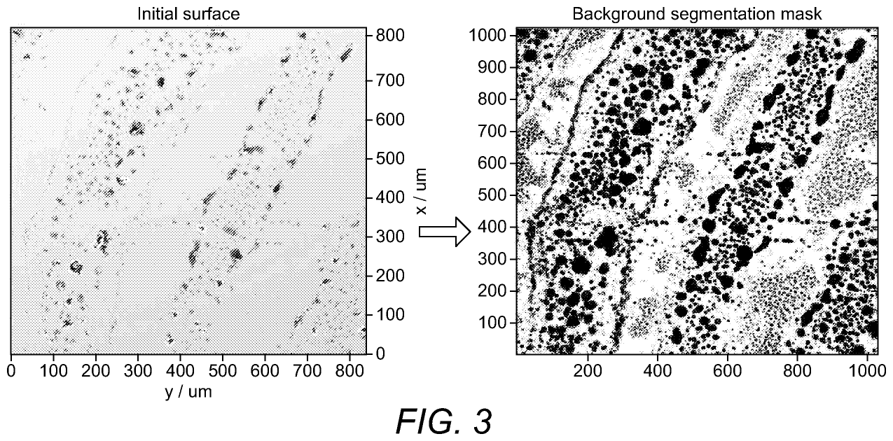

An example of segmentation using this threshold applied to a field of view containing a skin-print is shown in FIG. 3. The right hand figures distinguishes between regions of substrate in white and regions containing skin-print in black.

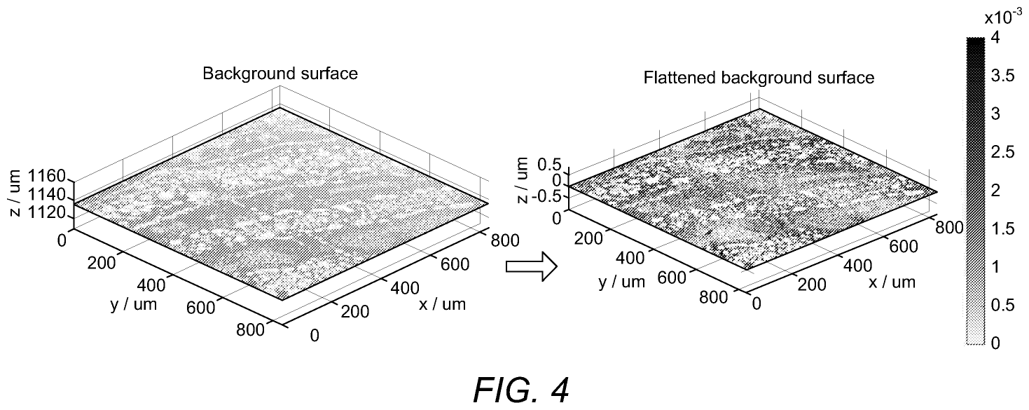

Next, in order to capture skin-print features that may have been misidentified as substrate, a second order polynomial least squares fit of the substrate points is subtracted from the surface, producing a flattened background surface. An example is shown in FIG. 4 wherein the left view shows before and the right view shows after the second order polynomial least squares removal of points corresponding to the substrate.

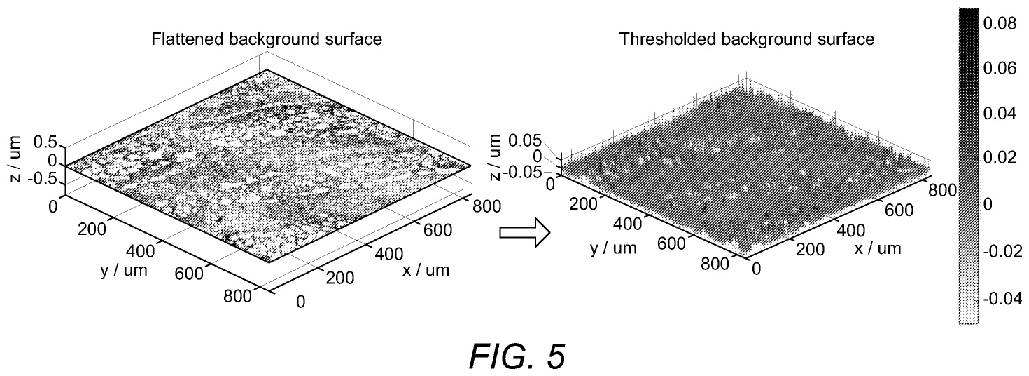

Subsequently, the surface may be thresholded between 0.5% and 99.5% of its vertical point values, under the supposition that this will cut out a majority of the non-substrate features misidentified as substrate. This step is shown in FIG. 5 (left before, right after).

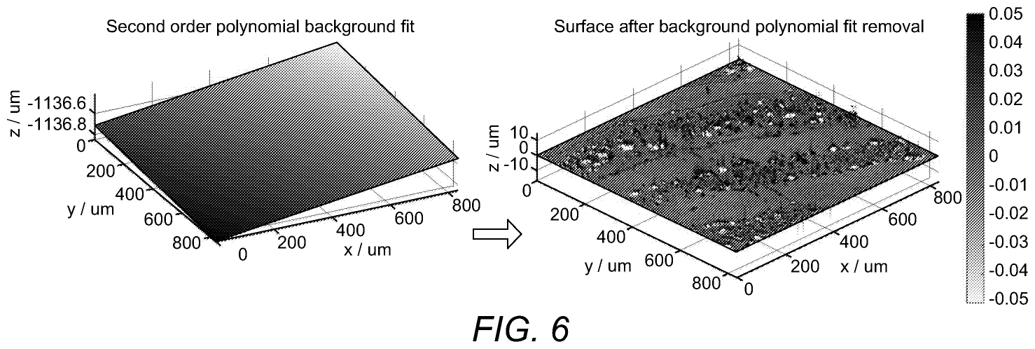

With this second segmentation of the background, a new background fit for the original surface is calculated and the background removal process is complete. This is shown in FIG. 6 (left: second order polynomial background fit; right: surface after removal of background polynomial fit).

Using a second order polynomial fit provides a good balance between processor time and accuracy of result. First, second and third order polynomial fits all provide results within nanometres of each other.

One issue inherent with the use of white light interferometry is that there is a limit on the features that are detectable where they involve steep slopes, or spikes. This occurs where light emitted by the white light interferometer is reflected away from its objective lens such that it is unmeasured by it. One approach to mitigating this may be to switch to an objective lens with a higher numerical aperture that accepts light from a wider range of angles, making it possible to measure features having steeper gradients. However, this will increase the measurement time and may still have limitations as to the measurable gradient.

Another issue that may occur involves features that scatter light multiple times before returning the signal to the objective. The resulting signal detected by the white light interferometer will have an interference pattern that may not be possible to process (returning an unmeasured data point) or may be resolved in to one or more heights not physically representative of the true height at a given point.

Accordingly, where features include a steep spike in topography, there may be non-measured data points. Also, where gradients are borderline too steep to be measured, or where diffusive material exists, this may result in erroneous height measurements.

Such erroneous height measurements may be caught by a filtering process that determines if the height at any given point makes physical sense when considering the adjacent data points and a physical understanding of the wetting characteristics of skin-print material.

One such filtering process to remove some of the poorly measured data points may be based on salt and pepper noise removal. A threshold value may be selected on the basis that a threshold exists beyond which a skin-print is not capable of physically supporting a structure having a specific topography. In one example, a threshold may be selected that is defined by a structure having a height of 1.5 μm but width of less than 0.816 μm (the width figure in this example corresponds to the lateral point spacing of the white light interferometry data).

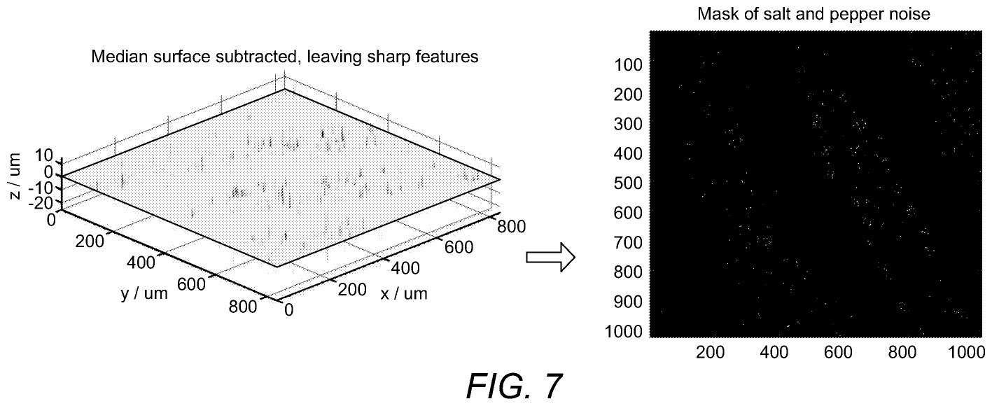

Based on this threshold, a salt and pepper mask may be deployed. The concept is illustrated in FIG. 7. The left side shows a the processed image prior to salt and pepper removal, while the right side show a mask on which the small number of data points that fall outside the threshold value are shown in white (against the other data points, representing virtually the whole area, shown in black).

Any missing data points, either directly unmeasured of those removed by the filtering process, may then be filled. To counteract the possibility that their absence does not cause a bias towards high or low volume values non-measured points may be filled in with an estimate of surface topography based on topography in the locality.

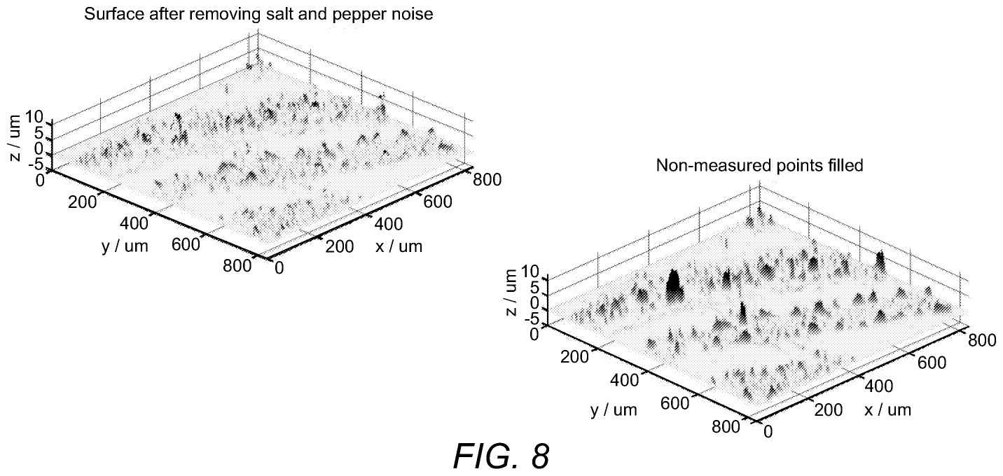

One simple approach may be to assume that the surfaces in the locality are such that they smoothly join the measured points along their boundaries to minimise or eliminate discontinuities. In other words, there may be smooth interpolation relative to the surrounding measured points. Such conditions may be satisfied by a model that requires the surface to satisfy a discrete form of the Laplace equation with Dirichlet boundary conditions provided by surrounding measured points. In one approach, such a model may be implemented using Matlab's “regionfill” function. An example is shown in FIG. 8 whereby the left view shows the surface with missing data points from directly unmeasured points and points removed in the filtering process while the right view shows the same surface with these missing points filled by smooth interpolation from the surrounding measured points.

In the event that all of the above processing is carried out on the data for each field of view in turn then the step of stitching the views together requires removal of the overlapping (5%) regions so as to avoid double counting volume along the edges of each field of view.

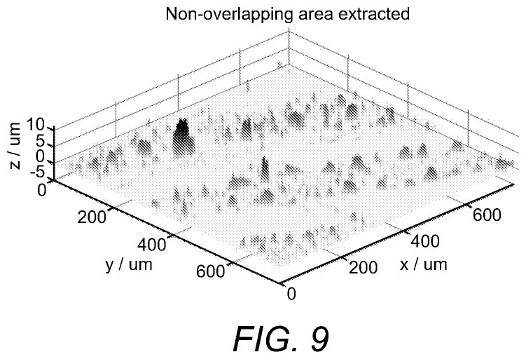

In some arrangements, this may be performed for each field of view prior to stitching. FIG. 9 shows a single field of view with the 5% duplicated data removed.

Having processed the data to maximise the accuracy of the topography of the skin-print and distinguished it from the underlying substrate, the volume of skin-print may then be calculated. This may be approximated as the volume bounded by the surface topography and the z=0 substrate plane. The volume from each point in the topography may be approximated as a cuboid using the lateral point spacing of 0.816 μm in the example above. The volume of each point may then be assumed to be 0.816 μm×0.816 μm×z μm. The volume of the print may then be obtained by summing the volume of each point across the wider area. Volumes may be calculated for each field of view sequentially and then summed over the grid to give overall volume.

As a final step, the individual views may be stitched in to a single data set that may then be used to assess the coverage of the skin-print residue and the local registration of each field of view. This stitched data set may also be used for traceability purposes linking the data set to an individual.

1. A method of determining a volume of skin-print residue deposited on a substrate, the method comprising:
performing interferometry on the substrate with skin-print residue deposited thereon to determine raw topography data of the substrate including the skin-print residue;
processing the raw topography data including subtracting topography of the substrate without the skin-print residue from the raw topography data in order to determine skin-print residue topography data;
determining volume of skin-print residue deposited on the substrate from the skin-print residue topography data.

2. The method of claim 1 wherein the interferometry comprises white light interferometry.

3. The method of claim 1 wherein the substrate comprises a skin-print deposition region having a first area and the method of performing interferometry involves an interferometric field of view having a view area that is smaller than the first area.

4. The method of claim 3 wherein the method of performing interferometry comprises performing interferometry sequentially on multiple view areas of the skin-print deposition region of the substrate in order to obtain interferometric data covering the full skin-print deposition region.

5. The method of claim 3 wherein each of the multiple view areas overlaps with at least one neighbouring view area and preferably wherein each of the multiple view areas overlaps with all immediately neighbouring view areas.

6. The method of any of claim 3 further comprising stitching together view area data.

7. The method of claim 5 further comprising stitching together view area data, wherein stitching together view area data comprises confirming relative positions by reconciling overlap between neighbouring view areas.

8. The method of claim 6 wherein stitching together view area data comprises enabling smooth joins along boundaries without discontinuities.

9. The method of claim 6 comprising removal of overlap between neighbouring view areas either before, after or as part of stitching together view area data.

10. The method of claim 1 wherein a total area covered by all multiple view areas is a subset of the full skin-print deposition region and data for areas falling outside that subset may be inferred.

11. The method of claim 10 wherein the subset comprises a checkerboard arrangement of view areas across the skin-print deposition region.

12. The method of claim 11 wherein the checkerboard arrangement covers half or one quarter or one eighth or one sixteenth of the area of the skin-print deposition region.

13. The method of claim 1 wherein the step of processing the topography data in order to determine skin-print residue topography data by subtracting topography of the substrate without the skin-print residue from the raw topography data comprises:
determining topography of the substrate without the skin-print residue.

14. The method of claim 13 wherein the step of determining topography of the substrate without the skin-print residue comprises:
determining underlying gradient of the substrate without the skin-print residue.

15. The method of claim 1 wherein the step of processing the raw topography data includes identifying one or more non-measured data points.

16. The method of claim 1 wherein the step of processing the raw topography data includes performing a polynomial regression analysis on the data.

17. The method of claim 16 wherein the polynomial regression analysis uses a second order polynomial.

18. The method of claim 16 wherein the polynomial regression analysis uses a second order polynomial least squares fit subtracted from the surface.

19. The method of claim 1 comprising identifying topographical features wherein height and gradient which skin-print residue would not be physically capable of supporting on the basis of height and gradient thresholds and identifying them as erroneous data points.

20. The method of claim 19 further comprising implementing a subtraction mask to remove the erroneous data points and optionally replacing them with a non-measured data point.

21. The method of claim 15 further comprising:
inferring a non-measured data point.

22. The method of claim 21 wherein the step of inferring a non-measured data point using topography data from an area surrounding the non-measured data point.

23. The method of claim 21 wherein the step of inferring a non-measured data point using topography data from an area surrounding the non-measured data point involves inferring data so as to provide smooth joins along boundaries without discontinuities.

24. The method of claim 21 wherein the step of inferring a non-measured data point involves obtaining a surface that satisfies a discrete form of the Laplace equation with Dirichlet boundary conditions provided by surrounding measured points.

25. The method of claim 1 wherein the step of processing the raw topography data includes using a median filter to identify sharp features.

26. The method of claim 25 wherein the step of processing the raw topography data comprises compensating for the sharp features.

27. The method of claim 1 wherein the volume across the substrate is determined by calculating volume for each field of view and summing over the grid representing the full substrate.

28. The method of claim 23 wherein the volume at each data point is approximated as a cuboid having cross sectional area determined by lateral point spacing between adjacent data points and a height as determined by height of the white light interferometer topography for the data point in question.

29. The method of claim 1 wherein the interferometry comprises dual-polarising interferometry.

30. The method of claim 1 wherein the step of performing interferometry employs a calibrated white light interferometer that is calibrated using metrology standards that link the result to national units of measure.

31-32. (canceled)

33. The method of claim 1 further comprising selecting or preparing the substrate to optimise contact angle for deposition of the skin-print.

34. The method of claim 33 wherein the step of selecting or preparing the substrate to optimise contact angle for deposition of the skin-print comprises polishing of the substrate.
